(19) United States
(12) Patent Application Publication
(10) Pub. No.: US 2004/0043076 A1
(43) Pub. Date: Mar. 4, 2004

(54) Prolonged release microspheres for injection delivery and preparation method

(76) Inventors: Claire Dulieu, Angers (FR); Jo?euml;l Richard, Longue (FR); Jean-Pierre Benoit, Avrille (FR)

Correspondence Address: BURNS DOANE SWECKER & MATHIS L L P, POST OFFICE BOX 1404, ALEXANDRIA, VA, 22313-1404, US

(21) Appl. No.: 10/296,314

(22) PCT Filed: May 22, 2001
(86) PCT No.: PCT/FR01/01575

(30) Foreign Application Priority Data
May 23, 2000 (FR) 00/06587

(51) Int. Cl. A61K 38/28; A61K 38/21; A61K 9/16; A61K 9/50; A61K 38/19; A61K 38/24

(52) U.S. Cl. 424/490; 424/85.4; 424/85.1; 514/12; 514/3

(57) ABSTRACT

The subject of the present invention is microspheres intended to be administered by injection, comprising a protein active ingredient and an agent coating the active ingredient intended to prolong its release, free of any trace of organic solvent and obtainable according to a coating method involving bringing the active ingredient and the coating agent into contact, with stirring, in a supercritical fluid, the said coating agent being soluble in the supercritical fluid. The protein active ingredient is not denatured.

The present invention relates to the field of microparticles intended to be administered by the subcutaneous or intramuscular injection route.

There is currently a need for prolonged-release pharmaceutical formulations intended for the administration of proteins by injection, which are free of any trace of organic solvents.

Intense efforts have to be made to develop efficient novel systems for the administration of proteins. All the conventional techniques for preparing controlled-release injectable microparticulate systems, whether they be the preparation of microcapsules by the emulsion (oil/water)/solvent evaporation method (Hora et al., Pharm. Res., 7 (1990), 1190-1194; Jalil, R. et al., L. Microencapsulation, 7 (1990) 294-325), the coacervation method in organic phase (Ruiz et al., Pharm. Res., 7 (1990) 928-934; McGee, J. P. et al., J. Controlled Release, 34 (1995) 77-86) or by the double emulsion (water/oil/water)/solvent evaporation technique (Ogawa, Y et al., Chem. Pharm. Bull., 36 (1988) 1095-1103), lead to the use of organic solvents. These require steps for accurately controlling and measuring the levels of residual solvents in order to limit these levels and to avoid any harmful side effects on the patient. Furthermore, government authorities introduce strict standards for avoiding contamination of the environment with the organic solvents inherent to the methods of production and to limit or even eliminate the use of organic solvents in pharmaceutical compositions. Finally, in protein formulations, problems of denaturation induced by contact with solvents and undesirable phenomena of adsorption at solvents/water interfaces can occur.

The subject of application EP 257 368 is microspheres for parenteral administration containing a protein coated with a fatty substance or a wax intended to prolong the release of the said protein. They are obtained by nebulization of a formulation containing protein, a salt and a surfactant. The protein and the additives are mixed with the wax or with the fatty substance in the molten state before being subjected to nebulization. In the context of this method, the protein is exposed to high temperatures, of the order of 75 to 80° C., which are necessary to bring about the melting of the wax. The application of such temperatures causes substantial denaturation of the protein. The microspheres described in application EP 257 368 are intended for veterinary use and the formulated protein active ingredients are not expensive, such that their substantial denaturation during the method of preparation does not constitute a serious disadvantage. The teaching of this document is not suited to the formulation of protein active ingredients which are particularly heat-sensitive and whose production cost is such that activity must be integrally preserved.

The subject of the present invention is microspheres intended to be administered by injection, comprising a protein active ingredient and an agent coating the active ingredient intended to prolong its release.

The microparticles according to the invention, containing a protein active ingredient, are distinguishable from the microparticles of the prior art by their matrix structure and by the absence of any trace of organic solvent and by the fact that the protein active ingredient is not denatured.

The microparticles according to the invention are free of any trace of organic solvent and they can be obtained according to a coating method involving bringing the active ingredient and the coating agent into contact, with stirring, in a supercritical fluid, the said coating agent being soluble in the supercritical fluid. The protein active ingredient is insoluble in the supercritical fluid and it is not denatured.

The mean size of the microparticles according to the invention is between 0.1 and 150 μm.

Their content of active ingredient is between 0.5 and 50% by weight, preferably between 3 and 20% by weight.

It has been demonstrated that the use of a method of preparing microparticles by a technique using a supercritical fluid and a coating agent soluble in this fluid makes it possible to obtain microspheres with advantageous properties.

Application EP 706,821 describes the coating of particulate active substances in the case where the coating agent used is soluble in supercritical CO₂. After solubilization in supercritical CO₂, the coating agent is brought into contact with the protein to be coated in a closed reactor, with stirring. Pressure and temperature modifications in the latter lead to desolvation of the coating agent and therefore to its precipitation over the active substance. This method does not involve either organic solvent or water, and is carried out at a relatively low temperature. More precisely, the particles of active substance are suspended in the supercritical CO₂, and then the coating agent is dissolved in the suspension. The pressure and/or the temperature are then decreased in a controlled manner so as to reduce the solubility of the coating in the supercritical fluid and to cause the deposition of the coating at the surface of the particles of active substance. The layer of coating may be monomolecular or may be up to 100 μm thick. The size of the encapsulated particles is between 20 nm and 500 μm. The coating is a fatty substance or a biodegradable polymer which is soluble in supercritical CO₂. The deposition of the coating is carried out between 30 and 45° C., between 70 and 280 bar for 30 minutes to 4 hours, with stirring.

The carrying out of the method according to the invention consists in suspending an active ingredient, with stirring, in a supercritical fluid containing at least one coating agent dissolved therein, and then in modifying the pressure and/or temperature conditions of the medium in order to bring about the coacervation of the particles, by desolvation of the coating agent around particles of active ingredient, that is to say to bring about the coacervation of the particles by physicochemical modification of the medium. This method leads to matrix microparticles containing several particles of protein active ingredient.

It has been discovered in the context of the present invention that by adapting the value of certain parameters of the method, in particular the content of active ingredient equivalent to the active ingredient/coating agent ratio, the mode of stirring, and the coating agent/supercritical fluid ratio, microspheres with advantageous properties and which have a matrix structure are obtained.

The supercritical fluid preferably used is supercritical CO₂ (CO₂SC), the typical initial operating conditions for this method are about 30 to 45° C. and from 75 to 280×10⁵ Pa, although it is possible to use higher values of either of the two parameters or of both, provided of course that the higher values have no harmful or degrading effect on the active ingredient being coated, or on the coating agents.

This method involves suspending in an autoclave an active ingredient insoluble in the supercritical fluid, and then introducing into this autoclave the coating agent which is in the state of a solute in the supercritical fluid.

The pressure and/or the temperature are then modified so as to reduce the solubility of the coating agent in the fluid. Thus, the affinity of the coating agent for the active ingredient increases such that this coating becomes adsorbed around the active ingredient. Once this coating agent is deposited on the active ingredient, the autoclave is depressurized and the microparticles are recovered.

To carry out this method, the active ingredient to be coated is placed in an autoclave equipped with a stirrer and then the system is pressurized by introducing into the autoclave a fluid supplied under supercritical conditions. Finally, the coating agent(s) is(are) introduced into the autoclave and then the temperature and/or the pressure inside the autoclave is modified in a controlled and regulated manner so as to gradually reduce the solubility of the coating agent(s). When the solubility of this (these) coating agent(s) in the supercritical fluid decreases, it(they) precipitate(s) and the affinity of these agents for the surface of the active ingredient leads to their adsorption onto this surface. A variant of this method consists in placing the coating agent in the autoclave before introducing the active ingredient therein or alternatively in introducing the active ingredient therein and then a fluid capable of changing to the supercritical state. The pressurization of the autoclave in order to produce a supercritical fluid state will then cause the dissolution of the coating agent in the said supercritical fluid.

The stirring speeds may vary between 100 and 1000 revolutions/min, preferably between 150 and 450 rpm. The choice of stirring speed depends on the size of the reactor.

Such a stirring brings about the suspension of the active ingredient in the supercritical fluid when the latter is introduced. The supercritical conditions are brought about by a modification of the temperature and/or of the pressure inside the autoclave. Thus, the temperature of the autoclave is between 30 and 45° C. and the pressure is between 100 and 280×10⁵ Pa and preferably between 180 and 220×10⁵ Pa. The coating agent is introduced into the autoclave at the same time as the supercritical fluid or alternatively after introduction of the supercritical fluid into the autoclave. In any case, to ensure good solubilization of the coating agent in the supercritical fluid, the system is maintained at equilibrium, with stirring, adequate concentration of active ingredient and of coating agent is established according to the microparticle desired and this equilibrium is kept stirring for about one hour. The temperature and the pressure are then varied at a sufficiently low speed to completely transfer the coating agent(s) in the supercritical fluid to the surface of the active ingredient and the system is depressurized in order to isolate the microparticles which are removed from the autoclave. At the time of depressurization, the stirring speed may be preserved, reduced or stopped.

The concentration of coating agent in the supercritical fluid is preferably between 1.5 and 4.5 g/l, preferably equal to 2 g/l.

According to a preferred embodiment of the invention, a cylindrical insert is placed in the autoclave, and is screwed to the cover before closing. The supercritical fluid is preferably introduced through the top part of the insert after closing the autoclave.

This insert is advantageously equipped with two sinters allowing the inflow and outflow of the supercritical fluid. The insert is preferably provided with an annular sinter in its top part, and with a discoid sinter constituting the bottom of the said insert. The two sinters advantageously have a porosity less than the size of the microspheres which it is desired to prepare.

The insert makes it possible to recover the microspheres containing the active ingredient. At the end of the process, it is unscrewed and moved, optionally in a chamber containing an inert gas when the protein active ingredient is sensitive to moisture, and is inverted in order to recover the microspheres. It allows the use of an inert propellant gas to facilitate the recovery of the microspheres containing the active ingredient when the said active ingredient is sensitive to moisture.

The coating agent entering into the composition of the microspheres of the invention, optionally chosen for carrying out their method of preparation, may be a biodegradable polymer or a fatty substance.

The coating agent is particularly chosen from

biodegradable polymers and copolymers of α-hydroxycarboxylic acids, in particular homopolymers and copolymers of lactic and glycolic acids, more particularly PLA (Poly-L-lactide) and PLGA (Poly-Lactic-co-Glycolic Acid),

poly(ε-caprolactone) and its derivatives, poly(β-hydroxybutyrate), poly(hydroxyvalerate) and (β-hydroxybutyrate-hydroxyvalerate) copolymers, polymalic acid,

amphiphilic block polymers of the polylactic acid-polyethylene oxide type, biocompatible polymers of the polyethylene glycol type, polyethylene oxides, block copolymers of the polyethylene oxidepolypropylene oxide type,

polyanhydrides, polyorthoesters, polyphosphazenes, and mixtures thereof.

These polymers, chosen to be effective coating agents, have a molar mass greater than 10³ g/mol, preferably greater than 2×10³ g/mol and more particularly between 2×10³ and 2×10⁵ g/mol.

The polymer is chosen such that it is soluble in the supercritical fluid, by adapting in particular the particle size, the crystallinity, the weightaverage molecular mass, the chemical composition, the functionalization of the side and/or end groups and the acid value.

All these parameters are adjusted in order to obtain the desired solubility of the polymer in the supercritical fluid and/or to obtain the desired release profile for the protein active ingredient.

The coating agent is also chosen from fatty substances such as phospholipids, in particular phosphatidylcholine, phosphatidylglycerol, diphosphatidylglycerol, dipalmitoyl-phosphatidylcholine, dioleylphosphatidylethanolamine, dioleyl-phosphatidyl-choline, dimyristoyl-phosphatidylglycerol, or glycerides of C₁₀-C₁₈ fatty acids, mono-, di- or triglycerides and mixtures thereof, in particular C₈ to C₁₂ triglycerides such as triglycerides of capric and caprylic acids, triglycerides of myristic acid, palmitic acid, stearic acid and mixtures thereof, solid fatty acid esters, in particular C₈ to C₁₈ fatty acid esters such as ethyl palmitate, ethyl myristate, octyldodecyl myristate, preferably C₈ to C₁₂ fatty acid esters and mixtures thereof.

The coating agent may also be a mixture of one of the polymers and of one of the fatty substances mentioned above.

A particularly preferred coating agent in the context of the present invention is a Gélucire® (mixture of mono-, di- and triglycerides, of fatty acid esters and of polyethylene glycol).

According to a preferred embodiment, the coating agent is a Gélucire®, the temperature in the autoclave is of the order of 45° C., the pressure in the autoclave is of the order of 200 bar and the stirring speed is of the order of 450 rpm.

The protein active ingredient may be a protein or a peptide.

The proteins falling within the scope of the present invention are chosen from the parathyroid hormone related protein, growth hormone (GH), α-, β- or γ-interferons, α- or β-erythropoletin (EPO), granulocyte colony stimulating factor (GCSF), granulocyte macrophage colony stimulating factor (GMCSF), PACAP polypeptide (pituitary adenylate cyclase activating polypeptide), vasoactive intestinal peptide (VIP), thyrotropin releasing hormone (THR), corticotropin releasing hormone (CRH), arginine vasopressin (AVP), angiotensin, insulin, somatotropin, the HBS antigen of the hepatitis B virus, plasminogen tissue activator, the coagulation factors VIII and IX, glucosylceramidase, sargramostim, lenograstin, filgrastin, interleukin-2, dornase-α, molgramostim, PEG-L-asparaginase, PEG-adenosin deaminase, hirudin, eptacog-α (human blood coagulation factor VIIa) and nerve growth factors (NGF, CNTF, BDNG, FGF, GDNF).

A particularly preferred protein in the context of the invention is erythropoietin, a glycosylated protein hormone which has a haematopoietic growth factor action. It is produced by genetic engineering under the name epoetin, and used clinically to maintain or raise the level of the patient's red blood cells. It is indicated in anaemia cases, during haemodialysis in chronic renal insufficiency sufferers, in parallel with a chemotherapy, in HIV patients or before a surgical operation. The treatment requires at least three injections per week.

Another particularly preferred protein in the context of the invention is alpha-interferon. Its activity spectrum is very broad since it is used both for its antiviral, anticancer and immunomodulatory properties. It is in particular used for the treatment of hepatitis B, hepatitis C, some leukaemias and Kaposi's syndrome. The treatment comprises three injections per week for 6 to 12 months.

Peptides, such as the derivatives of LHRH or of somatostatin, triptorelin, bombesin, calcitonin, parathyroid hormone, gastrin releasing peptide (GRP), luteinizing hormone releasing hormone (LHRH), growth hormone releasing factor (GRF), the peptide derivative Acetyl-Ser-Asp-Lys-Pro and amylin can also be used as active ingredient in the context of the present invention.

The size of the particles of protein active ingredient entering into the composition of the microspheres is between 20 nm and 60 μm, preferably between 15 and 50 μm.

The subject of the present invention is also a method of preparing microspheres as described above.

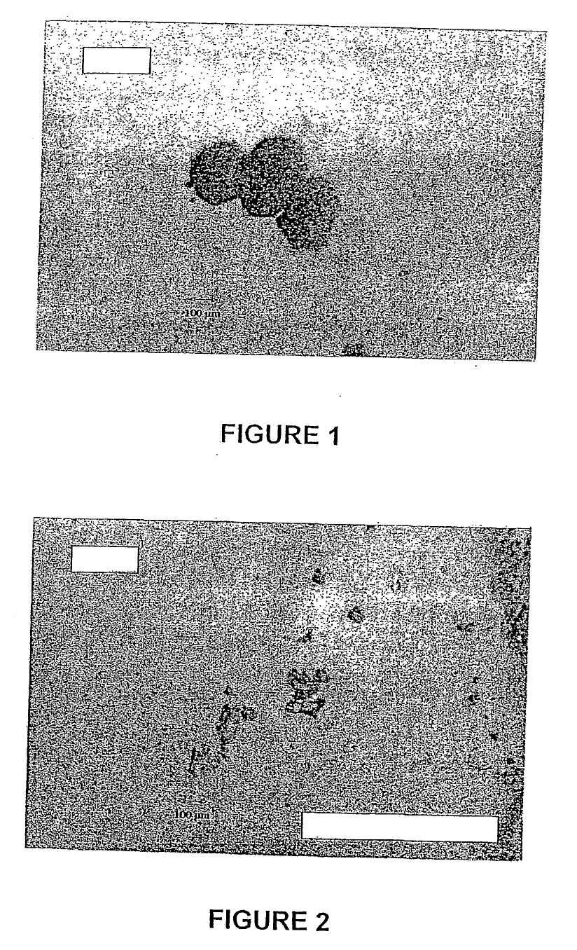

FIG. 1 is an optical micrograph of three microspheres obtained according to Example 1.

FIG. 2 makes it possible to demonstrate the matrix structure of the microspheres of FIG. 1. After addition of a few drops of dichloromethane (solvent for the coating agent), about fifteen crystals of BSA (insoluble in dichloromethane) are visible.

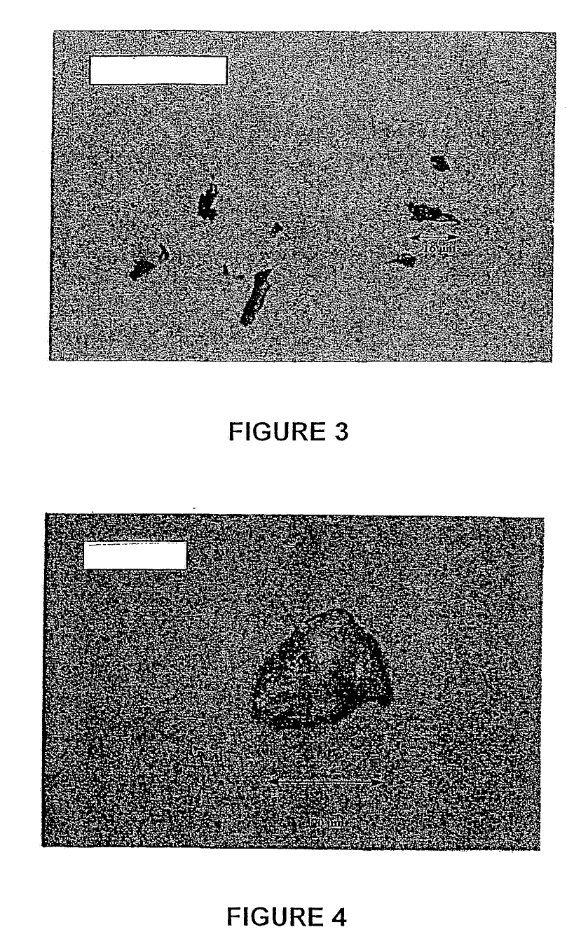

FIG. 3 is an optical micrograph of erythropoietin particles used in Example 2, before their coating.

FIG. 4 is an optical micrograph of a microsphere containing particles of erythropoietin obtained according to Example 2.

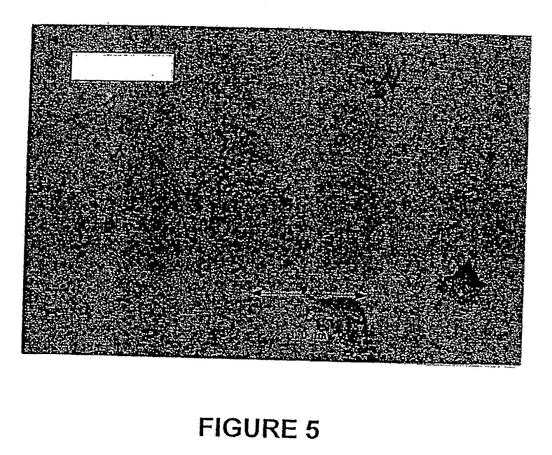

FIG. 5 makes it possible to demonstrate the matrix structure of the microsphere of FIG. 4. After addition of a few drops of dichloromethane (solvent for the coating agent), three crystals of erythropoietin are visible.

### EXAMPLE 1

### Microspheres of Gélucire® 50/02 Containing the Model Protein Bovine Serum Albumin (BSA)

Materials

300 ml autoclave provided with an insert 180 ml in volume, which is porous (10 μm) at the top and at the bottom. An autoclave provided with a jacket for the regulation of temperature: circulation of silicone oil, heated or cooled by a thermostatted bath. Stirring in the autoclave: motor controlled by PID control, shaft with a marine anchor-shaped twin blade rotor.

Products

BSA Fraction V (Sigma A-7906) ground in a mortar and sieved. 32 to 50 μm fraction: 118.45 mg.

Gélucire® 50/02 (Gattefosse), waxy mass reduced to chips with a spatula: 452.8 mg.

Procedure

The two products are placed at the bottom of the insert. The autoclave is closed and placed under stirring with 495 rpm. The successive sequences of formation of the microspheres are then the following:

injection of CO₂ by equilibration with the reservoir and then the column up to the initial conditions of 24.9° C. (T) and 99 bar.

heating of the autoclave by circulation of hot water in the jacket. The heating is regulated by PID; Duration of the heating 34 min.

maintaining equilibrium of the temperature/pressure parameters at 45.1° C. and 183 bar. These conditions are maintained for 66 min, stirring 495 rpm.

cooling by circulation of fluid in the jacket. Duration 41 min, up to: T=20° C., P=79 bar.

decompression in a buffer container to: T=25° C., atmospheric P. Duration=7 min.

The coated particles are recovered in the insert. About 150 mg thereof are recovered.

### EXAMPLE 2

### Microspheres of Gélucire® 50/02 Containing a Commercial Preparation of Solid Erythropoietin (Hémax®)

Material

Identical to that used in Example 1.

Products

Hémax® 2000 IU: 64 mg.

Gélucire® 50/02 (Gattefossé) waxy mass reduced to chips with a spatula: 259 mg.

Procedure

The two products are placed at the bottom of the insert. The autoclave is closed and placed under stirring with 460 rpm. The successive sequences of formation of the microspheres are then the following:

injection of CO₂ by equilibration with the reservoir and then the column up to the initial conditions of T=23° C. and P=100 bar.

heating of the autoclave by circulation of hot water in the jacket. The heating is regulated by PID. Duration of the heating 24 min.

stabilization of the temperature/pressure at 45° C. and 203 bar. These conditions are maintained for 65 min, stirring 460 rpm.

cooling by circulation of fluid in the jacket. Duration 49 min, to: T=17° C., P=74 bar.

decompression through vent opening to: T=25° C., atmospheric P. Duration=25 min.

recovery of the particles under a nitrogen atmosphere. 150 to 160 mg thereof are recovered.

### EXAMPLE 3

### Microspheres of Witepsol® E85 Containing a Commercial Preparation of Solid Erythropoietin (Hémax®)

Material

Identical to that used in Example 1.

Products (Quantities)

Hémax® 2000 IU: 150.2 mg

Witepsol® E85 (Condéa) C₁₀-C₁₈ fatty acid glyceride, waxy mass reduced to chips with a spatula: 450.2 mg.

Procedure

The two products are placed at the bottom of the insert. The autoclave is closed and placed under stirring with 460 rpm. The successive sequences of formation of the microspheres are then the following:

injection of CO₂ by equilibration with the reservoir and then with the column up to the initial conditions of T=22° C. and P=131 bar.

heating of the autoclave by circulation of hot water in the jacket. The heating is regulated by PID. Duration of the heating 46 minutes.

stabilization of the temperature/pressure at 35° C. and 199 bar. These conditions are maintained for 60 minutes, stirring 460 rpm.

cooling by circulation of cold water in the jacket. Duration 40 min, to: T=4.9° C., P=64 bar.

decompression through vent opening to 25° C. and atmospheric P. Duration 21 min.

recovery of the particles under a nitrogen atmosphere.

306.2 mg of a fluid powder of microparticles containing the protein are recovered.

### EXAMPLE 4

### Microspheres of Suppocire® ND Containing a Commercial Preparation of Solid Erythropoietin (Hémax®)

Material

Identical to that used in Example 1.

Products (Quantities)

Hémax® 2000 IU: 173.3 mg

Suppocire® ND (Gattefossé) mixture of mono-, di- and triglycerides, waxy mass reduced to chips with a spatula: 693.76 mg.

Procedure

The two products are placed at the bottom of the insert. The autoclave is closed and placed under stirring with 460 rpm. The successive sequences of formation of the microspheres are then the following:

injection of CO₂ by equilibration with the reservoir and then with the column up to the initial conditions of T=22° C. and P=129 bar.

heating of the autoclave by circulation of hot water in the jacket. The heating is regulated by PID. Duration of the heating 26 min.

stabilization of the temperature/pressure at 35° C. and 200 bar. These conditions are maintained for 60 min, stirring 460 rpm.

cooling by circulation of cold water in the jacket. Duration 45 min, to: T 22° C., P=61 bar.

decompression through vent opening to atmospheric P. Duration=31 min.

recovery of the particles under a nitrogen atmosphere.

439.76 mg of a fluid powder of microparticles containing the protein are recovered.

### EXAMPLE 5

### Microspheres of Dynasan Containing a Model Bovine Serum Albumin (BSA) Protein

Material The 1 l autoclave provided with a jacket for regulating the temperature: circulation of hot or cold water using a thermostatted bath. Stirring in the autoclave, shaft with a rotor having the shape of a marine anchor.

Products

BSA Fraction V (Sigma A-7906) ground in a mortar and sieved. 50-125 μm fraction: 400.30 mg.

Dynasan® 114 (Condéa), myristic acid triglyceride: 840.49 mg.

Dynasan® 116 (Condéa), palmitic acid triglyceride: 700.6 mg.

Dynasan® 118 (Condéa), stearic acid triglyceride: 59.85 mg.

Procedure

The four products are placed at the bottom of the autoclave. The autoclave is closed and placed under stirring with 210 rpm. The successive sequences of formation of the microspheres are then the following:

injection of CO₂ by equilibration with the reservoir and then with the column up to the initial conditions of T=25° C. and P=134 bar.

heating of the autoclave by circulation of hot water in the jacket. The heating is regulated by PID. Duration of the heating: 23 minutes.

stabilization of the temperature/pressure at 45° C. and 250 bar. These conditions are maintained for 60 minutes, stirring 210 rpm.

cooling by circulation of cold water in the jacket. Duration 37 min, to: T=15° C., P=72 bar.

stoppage of stirring,

decompression through vent opening to atmospheric P. Duration=30 min.

recovery of the particles under a nitrogen atmosphere.

1.34 g of a fluid powder of microparticles containing the protein are recovered.

### EXAMPLE 6

### Microspheres of Gélucire® 50/02 Containing a Model Bovine Serum Albumin (BSA) Protein

Material

60 l autoclave provided with an insert 50 l in volume. An autoclave provided with a jacket for the regulation of temperature: circulation of hot or cold water by a thermostatted bath. Stirring in the autoclave, shaft with three rotors having the shape of a 4-blade propeller.

Products (Quantities)

BSA Fraction V (Sigma A-7906) ground in a mortar and sieved. Fraction 50-125 μm: 80 g.

Gélucire® 50/02 (Gattefosse), waxy mass reduced to chips with a spatula: 180 g.

Procedure

The two products are placed at the bottom of the insert. The autoclave is closed and placed under stirring with 150 rpm. The successive sequences of formation of the microspheres are then the following:

injection of CO₂ by equilibration with the reservoir and then with the column up to the initial conditions of T=23° C. and P=90 bar.

heating of the autoclave by circulation of hot water in the jacket.

stabilization of the temperature/pressure at 45° C. and 200 bar. These conditions are maintained for 60 minutes, stirring 150 rpm.

cooling by circulation of cold water in the jacket. Duration 50 min, to: T=18° C., P=63 bar.

stoppage of stirring,

decompression through vent opening to atmospheric P. Duration=6 h.

recovery of the particles under a nitrogen atmosphere.

120 g of a fluid powder of microparticles containing the protein are recovered.

1. Microspheres intended to be administered by the subcutaneous or intramuscular injection route, comprising a protein active ingredient and an agent coating the active ingredient intended to prolong its release,
characterized in that
they are free of any trace of organic solvent,
they can be obtained according to a coating method involving bringing the active ingredient and the coating agent into contact, with stirring, in a supercritical fluid, the said coating agent being soluble in the supercritical fluid, and
the protein active ingredient is not denatured.

2. Microspheres according to claim 1, characterized in that their mean size is between 0.1 and 150 μm.

3. Microspheres according to claim 1 or 2, characterized in that their content of active ingredient is between 0.5 and 50% by weight, preferably between 3 and 20% by weight.

4. Microspheres according to one of claims 1 to 3, characterized in that the protein active ingredient is a protein chosen from the parathyroid hormone related protein, growth hormone (GH), α-, β- or γ-interferons, α- or β-erythropoietin (EPO), granulocyte colony stimulating factor (GCSF), granulocyte macrophage colony stimulating factor (GMCSF), PACAP polypeptide (pituitary adenylate cyclase activating polypeptide), vasoactive intestinal peptide (VIP), thyrotropin releasing hormone (THR), corticotropin releasing hormone (CRH), arginine vasopressin (AVP), angiotensin, insulin, somatotropin, the HBS antigen of the hepatitis B virus, plasminogen tissue activator, the coagulation factors VIII and IX, glucosylceramidase, sargramostim, lenograstin, filgrastin, interleukin-2, dornase-α, molgramostim, PEG-L-asparaginase, PEG-adenosin deaminase, hirudin, eptacog-a (human blood coagulation factor VIIa) and nerve growth factors (NGF, CNTF, BDNG, FGF, GDNF).

5. Microspheres according to claim 4, characterized in that the active ingredient is erythropoietin.

6. Microspheres according to one of claims 1 to 3, characterized in that the protein active ingredient is a peptide chosen from the derivatives of LHRH or of somatostatin, triptorelin, bombesin, calcitonin, parathyroid hormone, gastrin releasing peptide (GRP), luteinizing hormone releasing hormone (LHRH), growth hormone releasing factor (GRF), the peptide derivative Acetyl-Ser-Asp-Lys-Pro and amylin.

7. Microspheres according to one of claims 1 to 6, characterized in that the coating agent is chosen from
biodegradable polymers and copolymers of α-hydroxycarboxylic acids, in particular homopolymers and copolymers of lactic and glycolic acids, more particularly PLA (Poly-L-lactide) and PLGA (Poly-Lactic-co-Glycolic Acid),
poly(ε-caprolactone) and its derivatives, poly(β-hydroxybutyrate), poly(hydroxyvalerate) and (β-hydroxybutyrate-hydroxyvalerate) copolymers, polymalic acid,
amphiphilic block polymers of the polylactic acid-polyethylene oxide type, biocompatible polymers of the polyethylene glycol type, polyethylene oxides, block copolymers of the polyethylene oxide-polypropylene oxide type,
polyanhydrides, polyorthoesters, polyphosphazenes, and mixtures thereof.

8. Microspheres according to one of claims 1 to 6, characterized in that the coating agent is chosen from fatty substances such as
phospholipids, such as in particular phosphatidylcholine, phosphatidylglycerol, diphosphatidylglycerol, dipalmitoyl-phosphatidylcholine, dioleyl-phosphatidylethanolamine, dioleyl-phosphatidylcholine, dimyristoyl-phosphatidylglycerol,
glycerides of C₁₀-C₁₈ fatty acids,
mono-, di- or triglycerides and mixtures thereof, in particular C₈ to C₁₂ triglycerides such as triglycerides of capric and caprylic acids, triglycerides of myristic acid, palmitic acid, stearic acid and mixtures thereof,
solid fatty acid esters, in particular C₈ to C₁₈ fatty acid esters such as ethyl palmitate, ethyl myristate, octyldodecyl myristate, preferably C₈ to C₁₂ fatty acid esters and mixtures thereof.

9. Microspheres according to claim 8, characterized in that the coating agent is a Gélucire®.

10. Microspheres according to one of claims 1 to 9, characterized in that a method of preparation comprises the following steps:
suspension and dissolution, with stirring, respectively of the active ingredient and of the coating agent in the supercritical fluid,
modification of the temperature and/or of the pressure in order to desolvate the coating agent in a controlled manner and to cause its coacervation on the active ingredient, the stirring being maintained.

11. Microspheres according to claim 10, characterized in that the concentration of coating agent in the supercritical fluid is between 1.5 and 4.5 g/l, preferably equal to about 2 g/l.

12. Microspheres according to claim 10 or 11, characterized in that the coacervation temperature is between 30 and 45° C., the coacervation pressure is between 100 and 280×10⁵ Pa, preferably between 180 and 220×10⁵ Pa, and the stirring speed is between 100 and 1000 rpm, preferably equal to 450 rpm.

13. Microspheres according to one of claims 10 to 12, characterized in that an insert is placed in the autoclave and that the suspension and dissolution of the active ingredient and of the coating agent are respectively carried out in the insert.

14. Microspheres according to claim 13, characterized in that the insert is provided with two sinters allowing the inflow and outflow of the supercritical fluid.

15. Method of preparing microspheres according to one of the preceding claims, comprising the following steps:
suspension and dissolution, with stirring, respectively of the active ingredient and of the coating agent in the supercritical fluid,
modification of the temperature and/or of the pressure in order to desolvate the coating agent in a controlled manner and to cause its coacervation on the active ingredient, the stirring being maintained.

16. Method of preparation according to claim 15, characterized in that the concentration of coating agent in the supercritical fluid is between 1.5 and 4.5 g/l, preferably equal to about 2 g/l.

17. Method of preparation according to claim 15 or 16, characterized in that the coacervation temperature is between 30 and 45° C., the coacervation pressure is between 100 and 280×10⁵ Pa, preferably between 180 and 220×10⁵ Pa, and the stirring speed is between 100 and 1000 rpm, preferably equal to 450 rpm.

18. Method of preparing microspheres according to one of claims 15 to 17, characterized in that an insert is placed in the autoclave and that the suspension and dissolution of the active ingredient and of the coating agent are respectively carried out in the insert.
